(19) United States
(12) Patent Application Publication
(10) Pub. No.: US 2012/0263694 A1
(43) Pub. Date: Oct. 18, 2012

(54) Intimate Lubrication Kit

(76) Inventors: Susan Mary Lennox, Alton (GB); Sarah Annabelle Brooks, Petersfield (GB)

(21) Appl. No.: 13/324,554

(22) Filed: Dec. 13, 2011

Related U.S. Application Data
(63) Continuation of application No. PCT/GB2010/000601, filed on Mar. 26, 2010.

(30) Foreign Application Priority Data
Apr. 1, 2009 (GB) 0905677.1

(51) Int. Cl. A61K 36/55; A61P 15/00; A61K 35/74

(52) U.S. Cl. 424/93.45; 424/768

(57) ABSTRACT

The kit comprises applicators of lubricating composition for use during the days up to and including ovulation and applicators of a slightly thicker composition for use just after ovulation.

The lubricating composition is neutral or slightly acid with a view to being sperm friendly. The thicker composition is slightly acid to mirror vaginal acidity and includes a moisturiser for vaginal friendliness. It is also a lubricant.

### CROSS REFERENCE TO RELATED APPLICATION

This application is a continuation of and claims the benefit of International Application No. PCT/GB2010/000601 having an international filing date of Mar. 26, 2010 under 35 U.S.C. §120, and which in turn claims priority to U.K. Patent Application No. 0905677.1 filed on Apr. 1, 2009.

### BACKGROUND OF THE INVENTION

1. Field of the Invention

The present invention relates to a intimate lubrication kit.

Couples can experience discomfort when making love. Often this is caused by vaginal dryness.

2. Description of the Related Prior Art

In International Patent Application No. WO 2006/092581 there is described and claimed—at least on entry into the UK regional phase No GB 2,437,903 A—

A lubricating composition comprising:
linseed extract and a further seed polysaccharide extract soluble in water,
a pH buffer and
at least one preservative for prevention of growth of micro-organisms.

In production, this composition has been acidic. Vaginal fluids are acid typically in the region of a pH of 4 to 5. However sperm can be incapacitated by acidity, in that they cease to swim, i.e. become immotile. Sperm can also lose viability if in an aqueous environment where the osmolality is either too high, hyper-osmotic (greater than 380 mosm) or too low hypo-osmotic (less than 280 mosm). Semen is normally has a pH between 7 & 8 and has an osmolality in the region of 320 mosm, ranging from 260 to 365 mosm.

vaginal acidity is advantageous in discouraging infections such as thrush (Candida albicans), Bacterial Vaginosis and sexually transmitted infections such as Trichomonas. These infections reduce ability to conceive.

There is a correlation between vaginal dryness and infertility.

We have sought to develop a lubricating composition to help men and women, who are trying to conceive, whether or not they are suffering from infertility. However the fundamental dichotomy of vaginal acidity not suiting the sensibilities of sperm remains.

In seeking to overcome the dichotomy, we have devised and invented a dual composition lubricant one composition being sperm friendly and the other composition being vagina friendly.

### SUMMARY OF THE INVENTION

The object of the present invention is to provide a lubrication kit including such compositions.

According to the invention there is provided an intimate lubrication kit comprising:
at least one first container containing an aqueous lubricating composition, which composition:
   provides lubricity;
   is slightly acid or substantially neutral or slightly alkaline and
   is substantially iso-osmotic with semen and
at least one second container containing an aqueous moisturising composition, which composition:
   provides moisturisation,
   provides lubricity and
   is slightly to moderately acidic.

By providing “lubricity” is meant providing lubrication in the manner that soapy water lubricates wet hands rubbed together which can then slip easily across each other.

By providing “moisturisation” is meant ability to transport moisture to human tissue.

Preferably the moisturising composition is substantially iso-osmotic with typical vaginal fluids.

It should be particularly noted that both the lubricating and the moisturising compositions can have both lubricating and moisturising properties. The terms “lubricating composition” and “moisturising composition” are used in accordance with the properties that they are primarily intended to contribute during their respective uses.

Whilst we can envisage that either or both of the compositions could be formulated with synthetic materials, such as hydroxyethylcellulose, we prefer to formulate them with naturally derived materials, i.e. materials which have not been chemically synthesised, at least as far as possible. Not least, we prefer to use organically derived materials, i.e. materials derived from plants and preferably plants not treated with synthetic chemicals, because they are less likely in our experience to contain impurities such as traces of solvents and catalysts used in their product. For this reason, we further prefer to use organically derived materials which have not been extracted utilising synthetic materials such as extraction solvents, for instance hexane. For instance where we use aloe vera, we prefer to use it dried as opposed to extracted. In particular we prefer to formulate the lubricating composition with either or both of linseed and a further plant polysaccharide.

Normally we would expect the compositions to be hydrophilic; however, for lubricity, we can envisage lubricating composition to incorporate emulsified organically derived hydrophobic constituents, namely beeswax, plant oils and butters. Nevertheless we prefer to avoid linseed oil as such on account of its odour on oxidisation.

By careful choice of the water source from which the compositions are prepared, it is expected that the lubricating composition at least will require little or no addition of acid or alkali to bring it to be slightly acid or substantially neutral or slightly alkaline. Similarly, the moisturising composition be mildly acidic without addition. If this cannot be achieved by use of hard or soft water, we prefer to use citric acid, lactic acid or hydrochloric acid if the respective composition is too alkaline or sodium or potassium hydroxide if too acid. Other common acids and alkalis are possible. To maintain neutrality and slight acidity, we prefer to incorporate pH buffers in the composition, such as phosphates.

Again by choice of the water and other substances, the compositions are expected to be substantially iso-osmotic to semen and the vaginal fluids. If the osmolality requires to be adjusted, this may be achieved by use of an organic solute such as a sugar or an inorganic salt such as sodium chloride.

Preferably the lubricating composition will have a pH of 5.5 to 9.0—more likely 7.0 to 8.5—and an osmolality of 200 to 400 mosm, typically 280-380 mosm. The pH is intended to be more sperm friendly than vaginal acidity in order to enhance sperm survival and availability for fertilisation, in particular at the fertile period. Similarly the osmolality is intended to be sperm friendly. Further we believe that a target viscosity for this composition is 10,000 to 15,000 centipoise, in order to facilitate love making even with vaginal dryness. However, in so far as non-Newtonian properties are advantageous, be they shear thinning and/or pseudo-plasticity and/or thixotropic, we expect that viscosity outside this range will be suitable on occasion.

Preferably the moisturising composition will have a pH of 3.0 to 5.5—more likely 3.0 to 4.5—and an osmolality of 100 to 400 mosm, typically 250-310 mosm. The pH is intended to restore temporary deviation from natural vaginal pH, the deviation being in the interests of sperm friendliness. The osmolality is typically lower than that of the lubricating composition for moisturising. Further we believe that a target viscosity for this composition is 12,000 to 30,000 centipoise. In other words not only will the moisturising composition be more acidic to the first's slight acidity/neutrality/slight alkalinity, and we expect it to have a marginally lower osmolality and slightly higher viscosity than the lubricating composition. Nevertheless, we aim for the moisturising composition to be a compromise in being a moisturiser and a lubricant for love making after ovulation at any time until the next fertile period.

We envisage that we can include lactobacillus in the moisturising composition to enhance it natural occurrence in the vagina.

Additionally we prefer to incorporate a muco-adherent in the moisturising composition. It is possible to incorporate one in the lubricating composition, but this property is preferably enhanced in the moisturising composition, in the interest of moisturisation.

Whilst the compositions can be provided in bottles, preferably the kit comprises a plurality of small containers of the lubricating composition and a few containers of the moisturising composition. More containers of the moisturising composition can be provided for promoting vaginal health and combating vaginal dryness. Preferably the containers are long necked single use containers, typically 40 mm to 60 mm.

The containers typically contain 3 ml to 7 ml.

### BRIEF DESCRIPTION OF THE DRAWINGS

To help understanding of the invention, a specific embodiment thereof will now be described by way of example and with reference to the accompanying drawings, in which:

FIG. 1 is a diagram of an intimate lubrication kit in accordance with the invention.

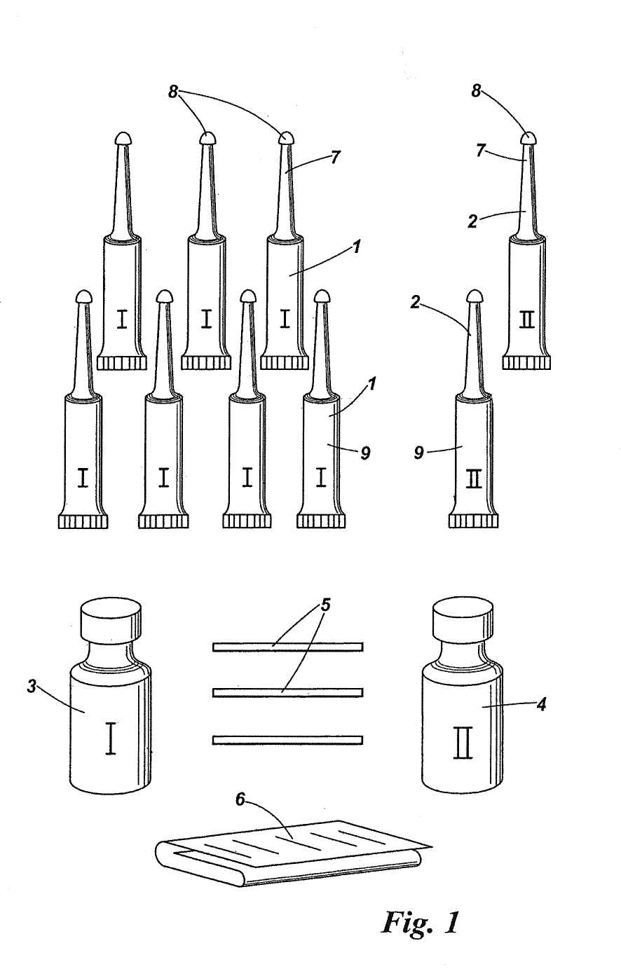

### DETAILED DESCRIPTION OF THE PREFERRED EMBODIMENT

Referring to the drawing, the intimate lubrication kit there shown has seven applicators 1 filled with lubricant and three applicators 2 filled with moisturiser. In addition, a bottle 3 of the lubricant and a bottle 4 of the moisturiser are provided. The kit also includes ovulation test strips 5 and instructions 6.

The applicators 1, 2 have long thin necks 7 with closure caps 8 and integral squeezable containers 9. The necks are typically 50 mm long and the containers typically have a 5 ml capacity. The bottles have 25 ml capacity.

Both the lubricant and the moisturiser are prepared in like manner to that described in International Patent Application No. WO 2006/092581 from a common aqueous mixture of linseed extract and Xanthan gum, with small quantities of lonicera caprifolium extract in the lubricant and potassium sorbate in the moisturiser as preservatives.

The lubricant has citric acid or sodium hydroxide added to adjust its pH to 7.0 to 8.5. Further sodium chloride is added to adjust its osmolality to 280-380 mosm. The moisturiser similarly has citric acid added to adjust its pH to 3.0 to 4.5 and its osmolality to 250-310 mosm. Further it has aloe vera extract added for its tissue benefits.

We believe these two compositions, i.e. the lubricant and the moisturiser, to be respectively sperm and vagina friendly.

The instructions for use are as follows:

Intimate Lubrication Kit

This kit is for couples experiencing difficulty in making love, in particular because of vaginal dryness. It may assist in conception.

The kit comprises 7 applicators of lubricating composition for use during the days up to and including ovulation and 3 applicators of a slightly thicker composition for use just after ovulation.

The lubricating composition is neutral or slightly acid with a view to being sperm friendly. The thicker composition is slightly acid to mirror vaginal acidity and includes a moisturiser for vaginal friendliness. It is also a lubricant.

The lubricating composition is applied to a couple's intimate organs when making love during the woman's most fertile period, i.e. typically the six days up to and including ovulation. Conveniently the composition can be applied to her vagina using the long necked applicators. The moisturising composition is applied to her vagina at least 24 hrs after ovulation is expected to have occurred.

Ovulation test strips are provided for assistance in determining the timing of ovulation.

Some couples may find it more comfortable to apply additional lubricant externally, either from one of the long necked applicators or from a separately provided bottle. Similarly, the moisturiser may be applied externally from the second long necked container or the bottle of moisturiser.

The invention is not intended to be restricted to the details of the above described embodiment. For instance, other aqueous polysaccharide aqueous extracts, for instance, Yellow Mustard seed gum, Acacia gum, gums from seaweeds (eg Alginates or Carrageenan), β-Glucans (derived from barley, oats, rye and wheat) and fruit gums. Preferred additional gums are Galactomannan gums, since they exhibit a synergistic phenomena resulting from their Galactomannan structures of long smooth sections of their molecules, for example Locust Bean gum, Guar gum, Cassia gum, Tara Gum, and Konjac Mannan gel which is a Glucomannan and is stereochemically similar to a Galactomannan. Certain of these gums are prepared in the manner of the linseed extract, for instance Yellow Mustard seed gum. Xanthan gum is prepared by action of the Xanthomonas campestris on maize seed. Further in place of potassium sorbate, Citricidal and/or phenoxyethanol may be used as preservatives

Whilst we prefer to use naturally occurring substances, we can envisage use of synthetic polysaccharides, Hydroxyethyl cellulose, glycerine, polyethylene oxide, polycarbophil, carbomers and/or glycols.

1. An intimate lubrication kit comprising:
at least one first container containing an aqueous lubricating composition, which composition:
provides lubricity;
is slightly acid or substantially neutral or slightly alkaline and
is substantially iso-osmotic with semen and
at least one second container containing an aqueous moisturising composition, which composition:
provides moisturisation,
provides lubricity and
is slightly to moderately acidic.

2. An intimate lubrication kit as claimed in claim 1, wherein the moisturising composition is substantially iso-osmotic with typical vaginal fluids.

3. An intimate lubrication kit as claimed in claim 1, wherein the moisturising composition has a lower pH than the lubricating composition.

4. An intimate lubrication kit as claimed in claim 1, wherein the lubricating composition and the moisturising composition contain substantially identical lubricity and moisturisation providing constituents.

5. An intimate lubrication kit as claimed in claim 1, wherein the lubricating composition has a comparatively lower viscosity, to enhance its lubricating properties with respect to the moisturising composition and the moisturising composition a comparatively lower osmolality, to enhance its moisturising properties with respect to the lubricating composition.

6. An intimate lubrication kit as claimed in claim 1, wherein the compositions are formulated with synthetic constituents.

7. An intimate lubrication kit as claimed in claim 1, wherein the compositions are formulated at least substantially with organically derived constituents.

8. An intimate lubrication kit as claimed in claim 7, wherein at least some of the organically derived constituents have been extracted without utilising synthetic materials.

9. An intimate lubrication kit as claimed in claim 7, wherein at least one of the organically derived constituents has been dried.

10. An intimate lubrication kit as claimed in claim 1, wherein the compositions contain either or both of an aqueous extract of linseed (flax) and a further seed polysaccharide.

11. An intimate lubrication kit as claimed in claim 1, wherein the compositions are hydrophobic.

12. An intimate lubrication kit as claimed in claim 11, wherein the lubricating composition contains emulsified organically derived hydrophobic constituents.

13. An intimate lubrication kit as claimed in claim 1, wherein the pH of the compositions derives from water used in their preparation.

14. An intimate lubrication kit as claimed claim 1, wherein the pH of the compositions is controlled by addition of selected ones of citric acid, lactic acid, hydrochloric acid, sodium hydroxide and potassium hydroxide.

15. An intimate lubrication kit as claimed in claim 1, wherein the compositions contain pH buffers.

16. An intimate lubrication kit as claimed in claim 1, wherein the compositions contain an organic solute or an inorganic salt for control of osmolality.

17. An intimate lubrication kit as claimed in claim 1, wherein the lubricating composition has a pH of 5.5 to 9.0 and an osmolality of 200 to 400 mosm.

18. An intimate lubrication kit as claimed in claim 17, wherein the lubricating composition has a pH of 7.0 to 8.5 and an osmolality of 280-380 mosm.

19. An intimate lubrication kit as claimed in claim 1, wherein the lubricating composition has a viscosity of 10,000 to 15,000 centipoise.

20. An intimate lubrication kit as claimed in claim 1, wherein the lubricating composition has non-Newtonian properties.

21. An intimate lubrication kit as claimed in claim 1, wherein the moisturising composition has a pH of 3.0 to 5.5 and an osmolality of 100 to 400 mosm.

22. An intimate lubrication kit as claimed in claim 21, wherein the moisturising composition has a pH of 3.0 to 4.5 and an osmolality of 250-310 mosm.

23. An intimate lubrication kit as claimed in claim 1, wherein the moisturising composition has a viscosity of 12,000 to 30,000 centipoise.

24. An intimate lubrication kit as claimed in claim 1, wherein the moisturising composition has non-Newtonian properties.

25. An intimate lubrication kit as claimed in claim 1, wherein the moisturising composition contains a muco-adherent or more thereof than the first composition.

26. An intimate lubrication kit as claimed in claim 1, wherein the lubricating composition contains lonicera caprifolium and/or the moisturising composition contains potassium sorbate as preservatives.

27. An intimate lubrication kit as claimed in claim 1, wherein the moisturising composition contains lactobacillus.

28. An intimate lubrication kit as claimed in claim 1, including a plurality of small containers of the lubricating composition and plurality of containers of the moisturising composition.

29. An intimate lubrication kit as claimed in claim 28, where the containers are long necked single use containers.

30. An intimate lubrication kit as claimed in claim 28, where the containers contain between 3 ml and 7 ml of their compositions.

31. An intimate lubrication kit as claimed in claim 1, including a bottle containing an additional quantity of the first lubricant.

32. An intimate lubrication kit as claimed in claim 1, including ovulation strips for determining the timing of ovulation.

33. An intimate lubrication kit as claimed in claim 32, including instructions for use of the lubricating composition for love making during the days up to and including ovulation and of the moisturising composition during love making and/or as a moisturiser use after ovulation at any time until the next fertile period.
